(19) United States
(12) Patent Application Publication
(10) Pub. No.: US 2016/0235298 A1
(43) Pub. Date: Aug. 18, 2016

(54) SYSTEMS AND METHODS FOR PRIMING AN INTRAOCULAR PRESSURE SENSOR CHAMBER

(71) Applicant: Novartis AG, Basel (CH)

(72) Inventor: Nicholas Max Gunn, Newport Beach, CA (US)

(21) Appl. No.: 15/138,385

(22) Filed: Apr. 26, 2016

Related U.S. Application Data
(63) Continuation of application No. 13/972,608, filed on Aug. 21, 2013.

(51) Int. Cl. A61B 3/16

(52) U.S. Cl. CPC A61B 3/16; A61F 9/00781

(57) ABSTRACT

An intraocular pressure monitoring and sensing device for implantation in an eye of a patient may include a substrate having a pressure sensor disposed on a top surface thereof and a pressure sensor cap disposed on the substrate over the pressure sensor. The pressure sensor cap may include a wall structure extending from the top surface of the substrate, the wall structure laterally surrounding the pressure sensor. The pressure sensor cap may further include a cap top situated above the pressure sensor, the cap top and wall structure together forming an interior chamber, and a chamber inlet providing fluid access to the interior chamber. At least one of the cap top and the wall structure includes a semi-permeable surface to aid in priming.

This application is a continuation of U.S. application Ser. No. 13/972,608 filed Aug. 21, 2013.

### BACKGROUND

The present disclosure relates generally to systems and methods for priming chambers within implantable devices that provide ophthalmic treatments. In some instances, embodiments of the present disclosure are configured to be part of an intraocular implant comprising at least a part of an intraocular pressure control system.

Glaucoma, a group of eye diseases affecting the retina and optic nerve, is one of the leading causes of blindness worldwide. Most forms of glaucoma result when the intraocular pressure (IOP) increases to pressures above normal for prolonged periods of time. IOP can increase due to high resistance to the drainage of the aqueous humor relative to its production. Left untreated, an elevated IOP causes irreversible damage to the optic nerve and retinal fibers resulting in a progressive, permanent loss of vision.

The eye's ciliary body continuously produces aqueous humor, the clear fluid that fills the anterior segment of the eye (the space between the cornea and lens). The aqueous humor flows out of the anterior chamber (the space between the cornea and iris) through the trabecular meshwork and the uveoscleral pathways, both of which contribute to the aqueous humor drainage system. The delicate balance between the production and drainage of aqueous humor determines the eye's IOP.

FIG. 1 is a diagram of the front portion of an eye that helps to explain the processes of glaucoma. In FIG. 1, representations of the lens 110, cornea 120, iris 130, ciliary body 140, trabecular meshwork 150, Schlemm's canal 160, and the edges of the sclera 170 are pictured. Anatomically, the anterior segment of the eye includes the structures that cause elevated IOP which may lead to glaucoma. Aqueous humor fluid is produced by the ciliary body 140 that lies beneath the iris 130 and adjacent to the lens 110 in the anterior segment of the eye. This aqueous humor washes over the lens 110 and iris 130 and flows to the drainage system located in the angle of the anterior chamber 180. The edge of the anterior chamber, which extends circumferentially around the eye, contains structures that allow the aqueous humor to drain. The trabecular meshwork 150 is commonly implicated in glaucoma. The trabecular meshwork 150 extends circumferentially around the anterior chamber. The trabecular meshwork 150 seems to act as a filter, limiting the outflow of aqueous humor and providing a back pressure that directly relates to IOP. Schlemm's canal 160 is located beyond the trabecular meshwork 150. Schlemm's canal 160 is fluidically coupled to collector channels (not shown) allowing aqueous humor to flow out of the anterior chamber. The sclera 170, the white of the eye, connects to the cornea 120, forming the outer, structural layer of the eye. The two arrows in the anterior segment of FIG. 1 show the flow of aqueous humor from the ciliary bodies 140, over the lens 110, over the iris 130, through the trabecular meshwork 150, and into Schlemm's canal 160 and out its collector channels.

As part of a method for treating glaucoma, a doctor may implant a device in a patient's eye. The device may monitor the pressure in a patient's eye and facilitate control of that pressure by allowing excess aqueous humor to flow from the anterior chamber of the eye to a drainage site, relieving pressure in the eye and thus lowering IOP. To exert appropriate control, an accurate measurement of the pressure about the patient's eye may be made. However, accurately monitoring the pressure in the eye or pressure around the eye poses a number of difficulties. For example, bubbles may form inside chambers used to measure the pressure at a remote location. These bubbles may degrade the accuracy of such measurements, in such a way that treatment is suboptimal.

The system and methods disclosed herein overcome one or more of the deficiencies of the prior art.

### SUMMARY

In one exemplary aspect, the present disclosure is directed to an intraocular pressure (IOP) sensing device for implantation in an eye of a patient. The IOP sensing device includes a pressure sensor, a substrate having the pressure sensor disposed thereon, and a pressure sensor cap disposed on the substrate over the pressure sensor. The pressure sensor cap includes a wall structure and a cap top. The wall structure extends from the top surface and laterally surrounds the pressure sensor. The cap top is situated above the pressure sensor, with the cap top and wall structure together forming an interior chamber. In the IOP sensing device, at least one of the cap top and the wall structure comprises a semi-permeable material. The IOP sensing device further includes a chamber inlet in the pressure sensor cap that provides fluid access to the interior chamber.

In yet another exemplary aspect, the present disclosure is directed to a method for priming a chamber in an IOP sensing device suitable for implantation next to an eye of a patient. The method includes steps of coupling a liquid source to the inlet of a pressure sensor cap and of beginning an injection of a liquid from the liquid source through the inlet and into an interior chamber of the pressure sensor cap. The interior chamber contains a gas that is displaced through a semi-permeable portion of the pressure sensor cap as the liquid is injected. The method further includes steps of detecting the displacement of all of the gas from the interior chamber and of stopping the injection of the liquid.

In yet another exemplary aspect, the present disclosure is directed to a method of fabricating a semi-permeable chamber in an IOP sensing device suitable for implantation next to an eye of a patient. The method includes steps of providing a substrate having a plurality of contacts thereon, of coupling a pressure sensor to the plurality of contacts, and of fixing a pressure sensor cap to the substrate. The pressure sensor cap forms an interior chamber that encloses the pressure sensor and that includes at least one semi-permeable surface. The method further includes a step of coupling a tube to an inlet of the pressure sensor cap.

It is to be understood that both the foregoing general description and the following drawings and detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following.

### BRIEF DESCRIPTION OF THE DRAWINGS

The accompanying drawings illustrate embodiments of the devices and methods disclosed herein and together with the description, serve to explain the principles of the present disclosure.

FIG. 1 is a cross-sectional diagram of the front portion of an eye.

FIG. 2 is a perspective view of an ocular implant device that carries an IOP sensing system according to exemplary aspects of the present disclosure.

FIG. 3 is a perspective view of an eye and an ocular implant device that includes an IOP sensing system according to exemplary aspects of the present disclosure.

FIG. 4A is a top view of an exemplary pressure sensor cap such as may be used in an IOP sensing system according to additional exemplary aspects of the present disclosure.

FIG. 4B is a cross-sectional view of the exemplary pressure sensor cap of FIG. 4A as seen along a line A-A according to exemplary aspects of the present disclosure.

FIG. 4C is a cross-sectional view of an alternative exemplary embodiment of an IOP sensing system according to exemplary aspects of the present disclosure.

FIGS. 5A, 5B, 5C, and 5D are cross-sectional views of the exemplary pressure sensor cap of FIGS. 4A and 4B undergoing a priming process according to exemplary aspects of the present disclosure.

FIG. 6A is a top view of an exemplary pressure sensor cap such as may be used in an IOP sensing system according to additional exemplary aspects of the present disclosure.

FIG. 6B is a cross-sectional view of the exemplary pressure sensor cap as seen along a line B-B of FIG. 6A according to exemplary aspects of the present disclosure.

FIG. 7A is a top view of an exemplary pressure sensor cap such as may be used in an IOP sensing system according to additional exemplary aspects of the present disclosure.

FIG. 7B is a cross-sectional view of the exemplary pressure sensor cap as seen along the line C-C of FIG. 7A according to exemplary aspects of the present disclosure.

FIG. 8 is a flowchart showing a method of priming a chamber in an intraocular pressure sensing device according to exemplary aspects of the present disclosure.

FIG. 9 is a flowchart showing a method of fabricating a semi-permeable chamber in an intraocular pressure sensing device according to exemplary aspects of the present disclosure.

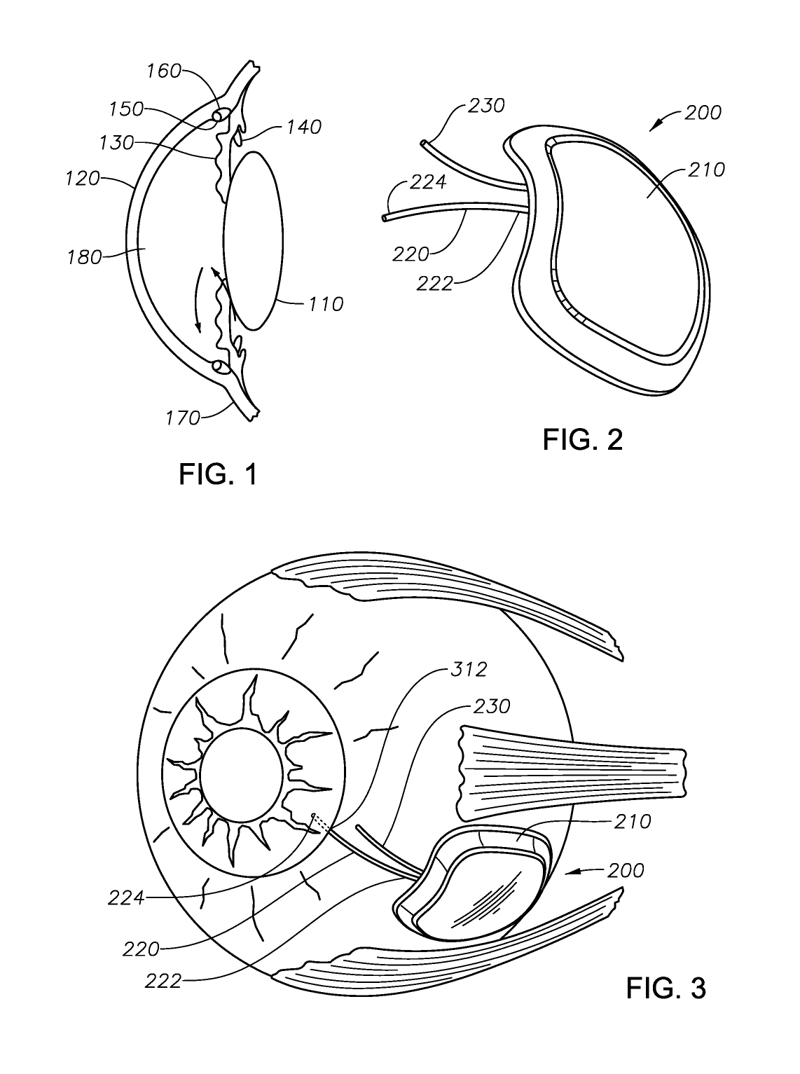

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The present disclosure relates generally to methods and systems for priming a chamber containing a pressure sensor for use in an intraocular pressure (IOP) monitoring device, such as a glaucoma drainage device (GDD). GDDs are used to alleviate excess pressure caused by aqueous humor accumulation in a patient's eye. The disclosed methods and systems may facilitate accurate pressure monitoring at a site removed from the pressure sensor by effectively purging air or another gas from a chamber containing the pressure sensor. Thus, the pressure measurement taken inside the chamber by the pressure sensor may more accurately correspond to the pressure at the site where the tube opening is placed. The systems and methods disclosed herein may thereby enable more accurate IOP determinations resulting in better information for determining treatment, potentially providing more effective treatment and greater customer satisfaction.

FIG. 2 is a schematic diagram of an intraocular implant or device 200 such as may be used in the monitoring and treatment of a patient's eye. As depicted, the intraocular device 200 is a GDD. The intraocular device 200 includes a body referred to herein as a plate 210 with a drainage tube 220 that extends from the plate 210. The drainage tube 220 includes a proximal end portion 222 that couples the tube to one or more structures internal to the plate 210. A distal end portion 224 of the drainage tube 220 may be coupled to the eye of a patient to allow for the monitoring of pressure and/or the drainage of fluid. As depicted, the intraocular device 200 includes an additional tube 230. The additional tube 230 may be used to provide atmospheric or ambient pressure measurements taken at a site close to the eye. It may provide access to a chamber that forms part of a IOP sensing system. This chamber will be discussed in greater detail below.

The plate 210 is configured to fit at least partially within the subconjunctival space and is sized within a range between about 15 mm×12 mm to about 30 mm×15 mm and has a thickness less than about 2 mm thick, preferably less than about 1 mm thick. The plate 210 may be formed to the radius of the eye globe (about 0.5 inches). It may be rigid and preformed with a curvature suitable to substantially conform to the globe or it may be flexible and can flex to conform to the globe. Some embodiments are small enough that conforming to the globe provides little benefit in comfort or implantation technique. The above dimensions are exemplary only, and other sizes and arrangements are contemplated herein. The plate 210 may include or be arranged to carry various components of an IOP control system. In some embodiments, such components include a power source, a processor, a memory, a data transmission module, and a flow control mechanism (i.e. valve system). It may also carry one or more pressure sensor systems.

FIG. 3 is a schematic diagram of an eye of a patient whose IOP is being monitored and/or who is receiving treatment with the intraocular device 200. In some embodiments, the drainage tube 220 extends from an anterior side of the plate 210 and is sized and arranged to extend into the anterior chamber of the eye through a surgically formed opening 312 in the sclera. The drainage tube 220 may be used to measure pressure in addition to facilitating drainage. In other embodiments, the drainage tube 220 and the additional tube 230 extend to other locations about the eye or body where multiple pressure measurements may be desired. The drainage tube 220 includes a first open end 224 that may be disposed at a location where pressure measurements may be desired, and at least one lumen that extends to a second open end 222 that may be disposed within or connected to the plate 210.

In some embodiments, the additional tube 230 may also extend from an anterior side of the plate 210 of the intraocular device 200. In such embodiments, the additional tube 230 may provide fluid access to a pressure sensor that measures pressure at an end of the tube. In one example, it measures the atmospheric pressure. An atmospheric reference pressure may be measured at a “dry” subconjunctival location. A “dry” location, as used herein, is a location spaced apart from an aqueous humor drainage site such that it is not influenced by the wetter tissue at the drainage site. This location may be covered and protected by a biocompatible patch material formed of, for example, donor sclera, pericardium, or others. Since atmospheric pressure is a factor used to determine IOP, the accuracy of the IOP measurement corresponds to the accuracy of the atmospheric pressure reading.

Prior to placement around a patient's eye as depicted in FIG. 3, one or more chambers within the plate 210 may be primed by the injection of liquid that displaces a gas from the chamber containing a pressure sensor. Liquid may be injected through the drainage tube 220 and/or the additional tube 230. Thus, in some embodiments, one or more chambers within plate 210 may be primed prior to positioning in or around a patient's eye.

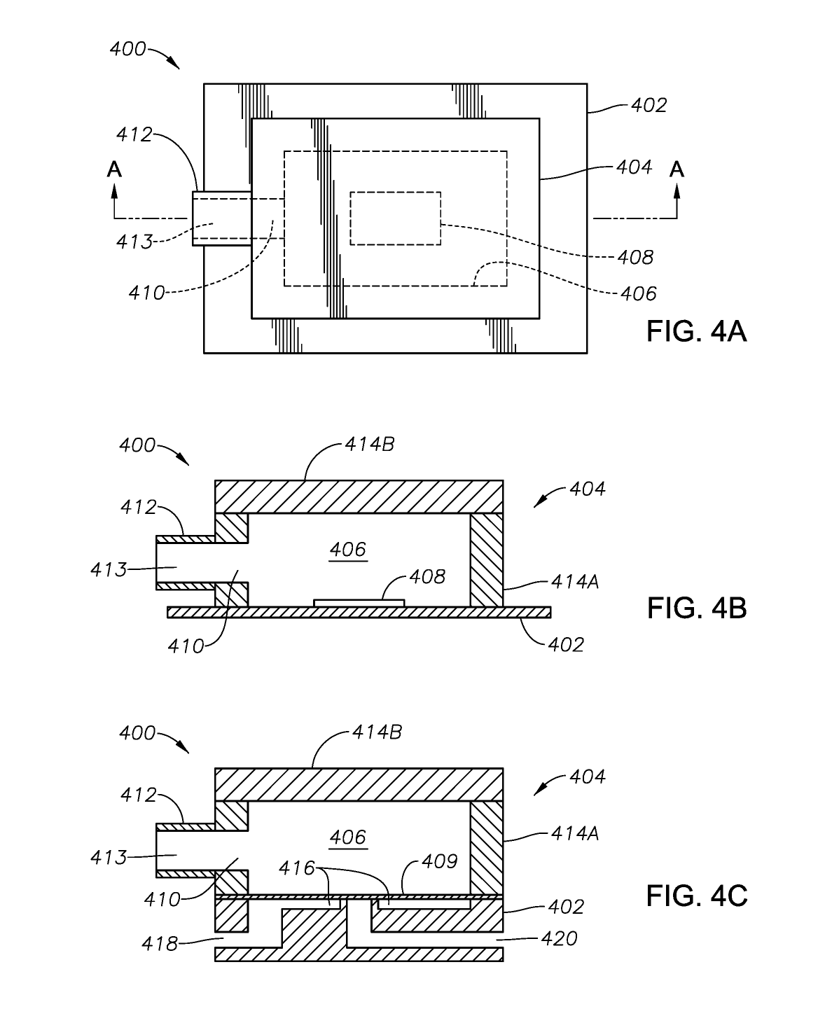

FIG. 4A illustrates a top view of an IOP sensing device 400. The IOP sensing device 400 includes a substrate 402 that may be formed from a printed circuit board material or other suitable material. While some features of the substrate 402 are depicted in FIG. 4A, many features are not explicitly depicted. For example, the substrate 402 may include a number of circuits, processors, power sources, and/or sensors with electrical leads both on a top surface of the substrate 402 and within it. In one embodiment, the substrate 402 is a flex circuit.

On top of the substrate 402 is a pressure sensor cap 404 that may be fixed on to the top surface of the substrate 402. The pressure sensor cap 404 cooperates with and is fixed to the substrate 402 to form an interior chamber 406. As depicted, the interior chamber 406 contains a pressure sensor 408. In other embodiments, additional sensors are positioned within the interior chamber 406 as well. For example, in some embodiments, the interior chamber 406 may also contain a temperature sensor and/or other sensors. The pressure sensor 408 may be electrically coupled to a plurality of leads within the chamber 406. In some embodiments, the pressure sensor 408 may have a ball grid array coupled to a plurality of contacts associated with the plurality of leads. In some other embodiments, the pressure sensor 408 may be wire bonded to a plurality of contacts within the chamber 406.

In order to allow access to the interior chamber 406 after the pressure sensor cap 404 is fixed to the substrate 402, an inlet 410 is provided in the pressure sensor cap 404. As depicted, the inlet 410 includes a protruding attachment member 412 having a lumen 413 extending therethrough. The lumen 413 further extends through the pressure sensor cap 404 such that gases, liquids, or other fluids may enter into the interior chamber 406. The attachment member 412 may facilitate the attachment and positioning of a flexible tube, such as a silicone tube. This flexible tube may be the drainage tube 220 or the additional tube 230 shown in FIGS. 2 and 3. Some embodiments of the IOP sensing device 400 may not include the attachment member 412. In such embodiments, a flexible tube may be abuttingly connected or insertably connected to the pressure sensor cap 404 using an adhesive and/or a press-fit connection.

FIG. 4B illustrates a cross-sectional view of the pressure sensor cap 404 as seen along line A-A of FIG. 4A. FIG. 4B thus provides additional perspective on the substrate 402, the interior chamber 406, the pressure sensor 408, and the inlet 410. Additionally, FIG. 4B shows that in some embodiments, the pressure sensor cap 404 may be formed from multiple subcomponents. As depicted, the pressure sensor cap 404 may include a wall structure 414A that extends up from the top surface of the substrate 402. Coupled to the wall structure 414A is a pressure sensor cap top 414B. The cap top 414B is positioned such that it is above the pressure sensor 408. In some embodiments, the wall structure 414A and the cap top 414B are formed separately and then joined together. In such embodiments, the wall structure 414A and the cap top 414B may be formed from different materials or from the same material. In other embodiments, the wall structure 414A and the pressure sensor cap top 414B are formed from a monolithic piece of material to provide the pressure sensor cap 404. The pressure sensor cap 404 may have an external area ranging from around 1 mm² to around 4 mm², with each side ranging in length from about 1 mm to about 2 mm.

Regardless of whether the wall structure 414A and the cap top 414B are formed from a single material or from different materials, the pressure sensor cap 404 includes a semi-permeable material. Thus, some embodiments of the pressure sensor cap 404 include a semi-permeable cap top 414B, other embodiments include a semi-permeable wall structure 414A, while in other embodiments both the cap top 414B and the wall structure 414A are semi-permeable. In yet other embodiments, only a portion of the wall structure 414A and/or the cap top 414B may be semi-permeable. While many different combinations of materials may be used to provide the pressure sensor cap 404, an exemplary embodiment may include a wall structure 414A formed from polyetheretherketone (PEEK) and a cap top 414B formed from polytetrafluoroethylene (PTFE), the PTFE acting as the semi-permeable material.

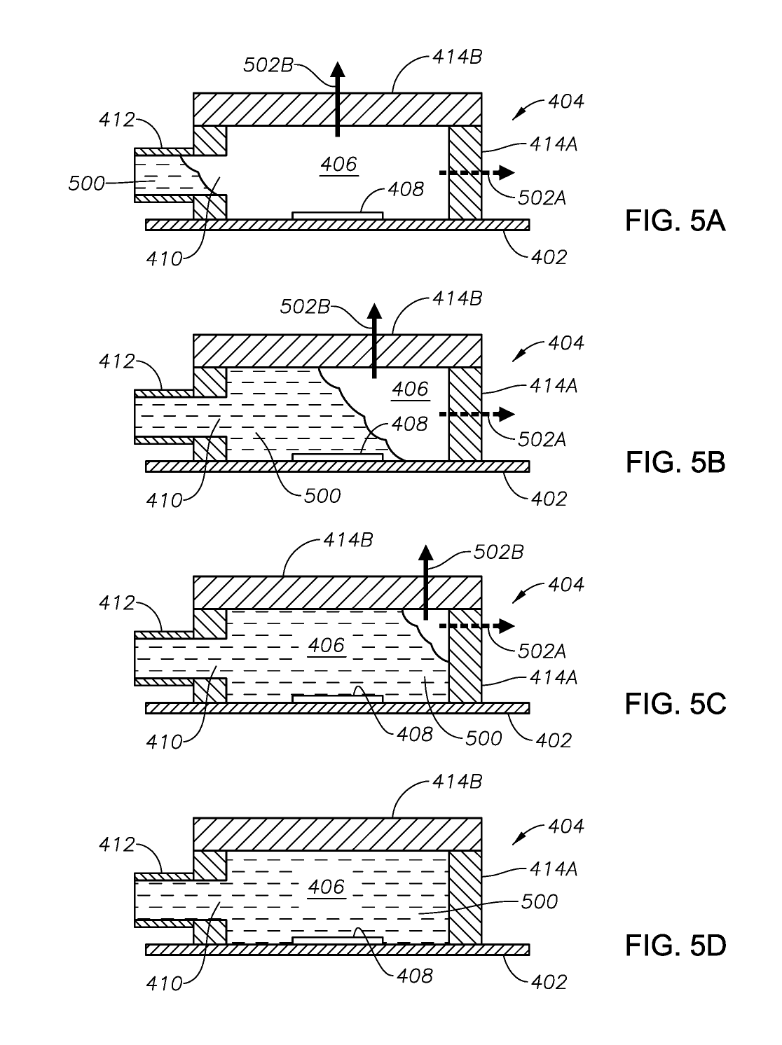

Other materials that may be used to create a semi-permeable pressure sensor cap 404 include high-density polyethylene, such as Tyvek® made by the E.I. du Pont de Nemours and Company of Wilmington, Del., polypropylene, and other materials. The permeability of material may be affected by pore size, hydrophobicity, and thickness. Some embodiments of the sensor cap 404 may range in thickness from about 0.1 millimeters to about 1 millimeter thick. Whether semi-permeable or not, the wall structure 414A and the cap top 414B may provide an adequate rigidity such that a pressure inside the interior chamber 406 and a pressure outside the chamber may be isolated from each other. Thus, it may be undesirable for the wall structure 414A or the cap top 414B to bend or flex significantly after positioning. The operation of the semi-permeable pressure sensor cap 404 may be better understood by reference to FIGS. 5A-D, discussed below.

FIG. 4C illustrates an alternate embodiment of the exemplary IOP device 400. Rather than include a pressure sensor 408 as depicted in FIGS. 4A and 4B, FIG. 4C includes a differential pressure sensor 409. As illustrated, the differential pressure sensor 409 is a mechanical differential pressure sensor. The differential pressure sensor 409 may be formed from a flexible member or membrane situated below the pressure sensor cap 404 and above the substrate 402. As depicted in FIG. 4C, the substrate is patterned to include a chamber 416, which has an inlet 418 and an outlet 420. The substrate 402 is patterned so that portions of the substrate 402 contact the membrane of pressure sensor 409 to create a seal under specific conditions. When the pressure within the chamber 406 is greater than a pressure within the chamber 416, the membrane of pressure sensor 409 and the portions of substrate 402 form and maintain a seal, such that a liquid is prevented from flowing from the inlet 418 to the outlet 420.

For example, the chamber 406 may be pressurized by the atmosphere, such that an atmospheric pressure is present within the chamber 406, and thus exerted on the membrane of 409 from above as viewed in FIG. 4C. The inlet 418 may be coupled to the anterior chamber 180 of an eye so that the pressure within the anterior chamber 180 is present within the chamber 416. When the atmospheric pressure is greater than the anterior chamber pressure, aqueous humor may be prevented from flowing out through the outlet 420. However, when the pressure present in the anterior chamber 180 is greater than the atmospheric pressure, or greater than the cumulative effects of the atmospheric pressure and an offset proportional to the mechanical and geometric characteristics of the substrate 402, the membrane 409, and/or other components, the membrane of the pressure sensor 409 may be displaced toward the cap top 414B, allowing aqueous humor to drain out through the outlet 420. In this manner, the pressure sensor 409 may measure and respond to differences in the pressures in chambers 406 and 416. The mechanical and geometric characteristics of the substrate 402 and the membrane of pressure sensor 409 may be selected so that the offset is a known, desired offset.

FIGS. 5A, 5B, 5C, and 5D illustrate cross-sectional views, as seen in FIG. 4B, of the exemplary IOP device 400 of FIG. 4A, undergoing a priming process. In order to prime the interior chamber 406 prior to implantation, a doctor or technician may couple one end of a tube to the attachment member 412 and the other end of the tube to a liquid source, such as a syringe, filled with saline or other such appropriate solution. As the doctor or technician manually exerts pressure on the syringe, the liquid from the syringe flows through the tube and into the inlet 410, as depicted in FIG. 5A. As the liquid 500 passes through the tube and into the inlet 410, the air that previously filled the tube is forced into the interior chamber 406. As the pressure inside the chamber 406 increases, the air may exit through the semi-permeable material of the pressure sensor cap 404. As depicted by an arrow 502A, if the wall structure 414A is semi-permeable, the air may escape through it. As depicted by an arrow 502B, if the cap top 414B is semi-permeable, the air may escape through it.

As depicted in FIG. 5B, as more liquid 500 is injected into the anterior chamber 406, more air is expelled through the semi-permeable material of pressure sensor cap 404. As in FIG. 5A, the air may exit the interior chamber 406 through the wall structure 414A and or the cap top 414B. This process may continue as seen in FIGS. 5C and 5D. As more liquid 500 is injected into the interior chamber 406 the gas that previously occupied the chamber may be forced through the semi-permeable material of the pressure sensor cap 404. The doctor who injects the liquid 500 may manually detect when the gas has been fully purged from the interior chamber 406, as depicted in FIG. 5D. This condition may be detected as the force required to depress the syringe tactilely increases, or as the syringe stops moving under a constant force. However, if excessive pressure is applied in injecting the fluid into the chamber 406, the liquid 500 may be forced through the semi-permeable material in some portion or portions of the sensor cap 404. This may damage the sensor cap 404.

In some embodiments, the pressure sensor 408 may be used during the priming process. In such embodiments, a completely primed state, such as depicted in FIG. 5D, may be detected by the pressure sensor 408 as a significant increase in pressure. In yet other embodiments, the priming may be performed in an automated process, in which a computer-controlled system injects the fluid until the significant increase in pressure occurs, at which point the computer-controlled system may stop the injection of liquid.

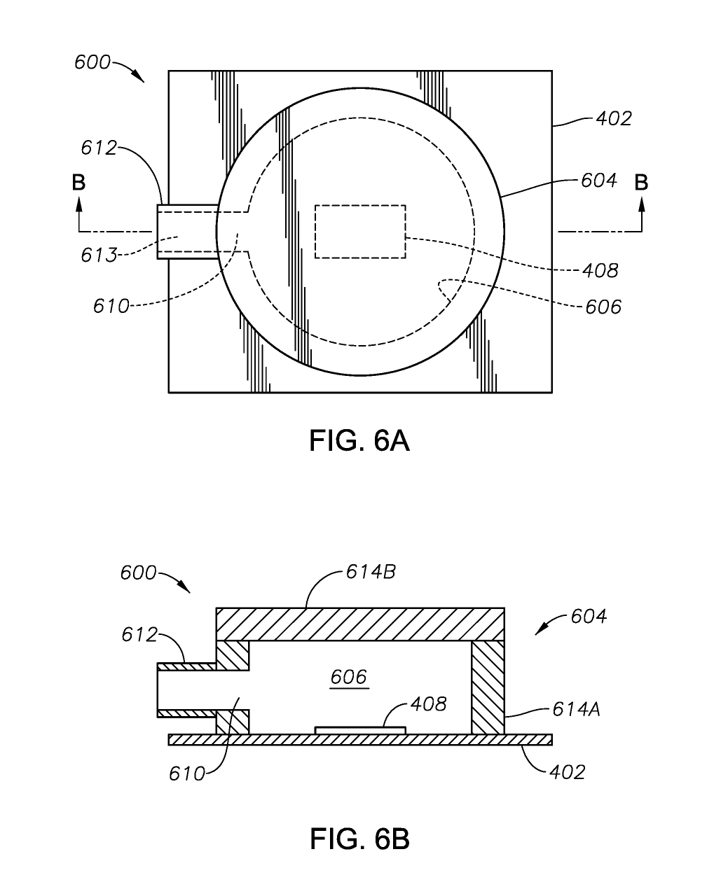

FIG. 6A is a top view of an exemplary IOP sensing device 600. The IOP sensing device 600 shares many similarities with the IOP sensing device 400 as described above and as depicted in FIGS. 4A, 4B, and 5A-D. The IOP sensing device 600 includes a substrate 402 with a pressure sensor cap 604 thereon. The pressure sensor cap 604 and the substrate 402 form an interior chamber 606, which may contain a pressure sensor 408. Some embodiments of IOP sensing device 600 may include a differential pressure sensor, such as pressure sensor 409 of FIG. 4C. Unlike the interior chamber 406 of FIGS. 4A-B and 5A-D, which as depicted has a rectangular cross-section as viewed from above, the interior chamber 606 as seen in FIG. 6A has a curved cross-section. As depicted, the interior chamber 606 has a circular shape, while other embodiments may have other elliptical shapes, or an ovoid shape. The elliptical shape of the interior chamber 606 may further inhibit the formation of trapped bubbles within the chamber. Also depicted in FIG. 6A, the IOP sensing device 600 includes an inlet 610 providing fluid access to the interior chamber 606, and an attachment member 612 having a lumen 613 extending therethrough. The attachment member 612 may not be present in some embodiments.

FIG. 6B is a cross-sectional view of the exemplary pressure sensor cap 604 as seen along line B-B depicted in FIG. 6A. FIG. 6B provides additional perspective on the substrate 402, the interior chamber 606, the pressure sensor 408, and the inlet 610. Additionally, FIG. 6B shows that in some embodiments, the pressure sensor cap 604 may be formed from multiple subcomponents. As depicted, the pressure sensor cap 604 may include a wall structure 614A that extends up from the top surface of the substrate 402. Coupled to the wall structure 614A is a pressure sensor cap top 614B. The cap top 414B is positioned such that it is above the pressure sensor 408. In some embodiments, the wall structure 614A and the cap top 614B may be formed separately and then joined together. In such embodiments, both the wall structure 614A and the cap top 614B may be formed from different materials or from the same material. Additionally, the wall structure 614A and the cap top 614B may be formed from a single piece of material, which may obviate a need to join two separate pieces of material. The pressure sensor cap 604 may have an internal surface area ranging from around 0.6 millimeters² to around 25 millimeters², with the diameter ranging in length from about 0.25 millimeters to about 5 millimeters. The IOP device 600 may be primed in a manner similar to that depicted in FIGS. 5A-5D and described above.

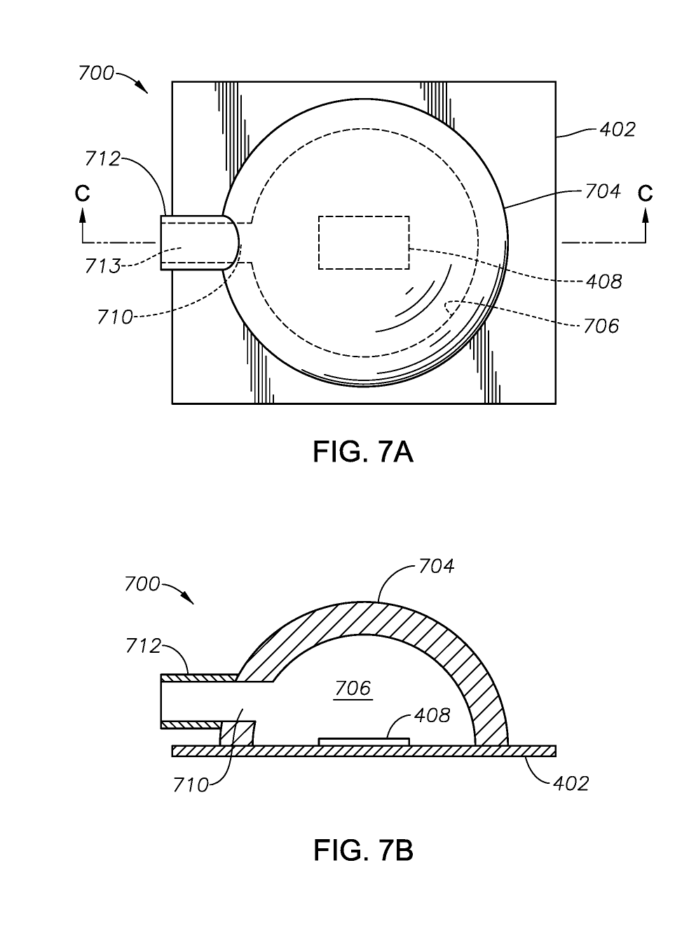

FIG. 7A is a top view of an exemplary pressure sensor cap 704 such as may be used in an IOP sensing device 700. The IOP sensing device 700 may share many features discussed above in connection with the IOP sensing devices 400 and 600. For instance, the IOP sensing device 700 includes a substrate 402, upon which the sensor cap 704 is fixed, forming an interior chamber 706 therebetween. The chamber 706 contains a pressure sensor 408. Some embodiments of IOP sensing device 700 may include a differential pressure sensor, such as pressure sensor 409 of FIG. 4C. As viewed from above, the pressure sensor cap 704 is approximately circular in shape; however other embodiments of the sensor cap 704 may have different shapes, such as rectangular, elliptical, etc. In order to provide access to the interior chamber 706, the sensor cap 704 includes an inlet 710 and an attachment member 712 having a lumen 713 extending therethrough. Although the attachment member 712 may facilitate the coupling of a tube to the pressure sensor cap 704, some embodiments of the IOP device 700 may not include the attachment member 712.

FIG. 7B is a cross-sectional view of the IOP sensing device 700 as seen alone the line C-C, depicted in FIG. 7A. FIG. 7B provides additional perspective on the features disclosed above. As depicted, the sensor cap 704 is approximately hemispherical in shape. This may further inhibit the formation of bubbles within the chamber during a priming process, such as that depicted in FIGS. 5A-5D. Embodiments of the pressure sensor cap 704 may have a diameter ranging from about 1 mm to about 5 mm. In the depicted embodiment, the pressure sensor cap 704 is formed from a monolithic piece of semi-permeable material. However, in other embodiments, more than one material may be used to form the cap 704. In such embodiments, only a portion of the pressure sensor cap 704 may be semi-permeable.

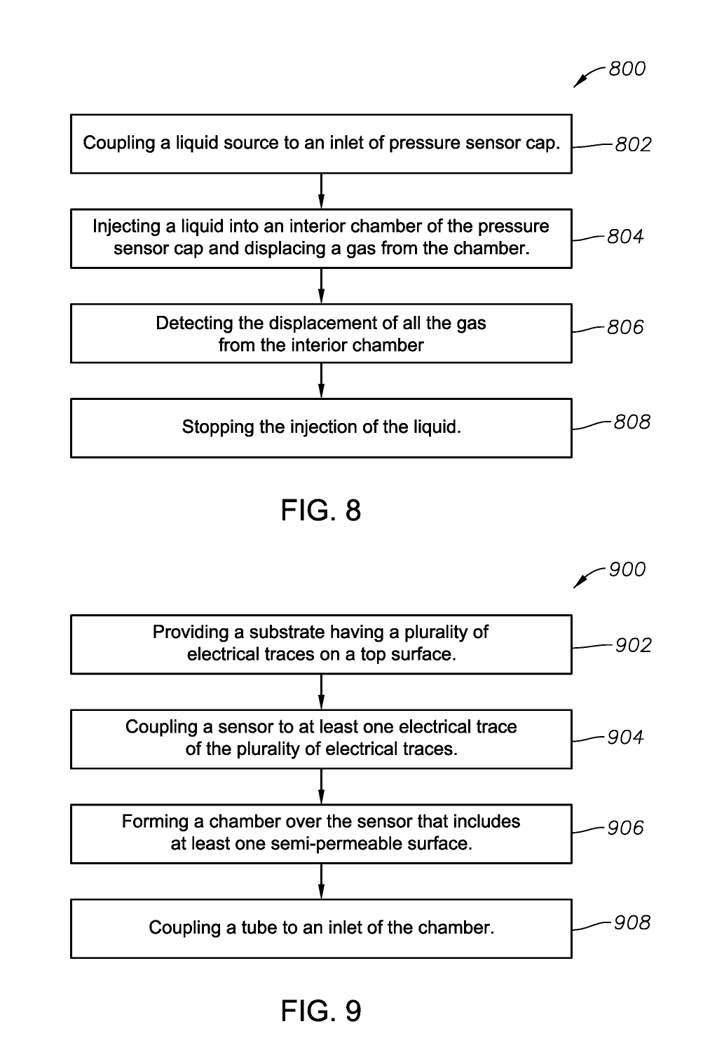

FIG. 8 shows a method 800 of priming a chamber in an intraocular device suitable for implantation next to an eye of a patient. As depicted, the method 800 includes a number of enumerated steps. However, embodiments of the method 800 may include additional steps before, in between, and after the enumerated steps. Method 800 begins at a step 802, when a liquid source is coupled to an inlet of a pressure sensor cap, the pressure sensor cap being included in the IOP sensing device. In step 804, a doctor or technician begins injecting a liquid from the coupled liquid source into an interior chamber, such that the liquid displaces a gas, which exits the chamber through a semi-permeable material of the pressure sensor cap. In some embodiments, a computer-controlled machine performs the injection. In step 806, the displacement of all the gas from the interior chamber is detected. And the injection of the liquid is stopped at step 808.

To better describe the method 800, reference is made herein to the IOP sensing device of FIGS. 4A-4B and 5A-5D. The method 800 may also be performed with other embodiments, including those depicted in FIGS. 6A-B and 7A-B. As depicted in FIGS. 4A and 4B, the IOP sensing device 400 includes an inlet 410, with an attachment member 412. The step 802 may be performed when a flexible tube (not shown) is coupled to the attachment member 412 on one end of the tube and to a liquid source, such as a syringe containing a liquid, on the other end of the tube. At step 804, the doctor or technician may begin injecting the liquid by manually actuating the syringe. In the computer-controlled embodiments, the machine may begin the injection using a pump or other flow driving system. As the liquid flows through the tube, through the inlet 410, and into the interior chamber 406, air that was present in the tube (not shown) and in the interior chamber 406 may be forced through the semi-permeable material or surface of the pressure sensor cap 404. Depending on the particular embodiment of the pressure sensor cap 404, the air may exit the interior chamber 406 through the wall structure 414A, the cap top 414B, or both.

As the liquid fills the interior chamber 406, the flow of liquid into the interior chamber 406 may be roughly consistent until the chamber is filled as seen in FIG. 5D. At step 806, when the chamber is filled, a change in flow may be observed by the doctor or technician, or by a machine, and the observation may be interpreted as an indication that all the gas is removed from the chamber. Additionally, a doctor or technician may determine that the gas has been removed when the force required to compress the syringe tactically increases. In some embodiments, the pressure sensor 408 may indicate an increase in pressure associated with the gas being completely purged from the interior chamber 406. At step 808, after the displacement of all the gas from the interior chamber 406, the doctor or technician, or controller in automated or semi-automated embodiments, may stop the injection and detach the liquid source from the flexible tube.

FIG. 9 shows a method 900 of fabricating a semi-permeable chamber in an IOP sensing device. As depicted, the method 900 includes a number of enumerated steps. However, embodiments of the method 900 may include additional steps before, in between, and after the enumerated steps. Method 900 begins at step 902 in which a substrate is provided. The substrate may include a plurality of electrical traces (not depicted) on a top surface thereof and/or contacts on the top surface that are in connection with electrical traces below the top surface. At step 904, a sensor is coupled to at least one electrical trace on the top surface of the substrate. At step 906, a chamber having at least one semi-permeable surface is formed over the sensor. The semi-permeable surface may allow passage of a gas therethrough, while blocking a liquid. At step 908, a tube (not depicted) is coupled to an inlet of the chamber.

In order to better describe method 900, reference is made herein to the IOP sensing device 400 of FIGS. 4A-B and 5A-D. A performance of method 900 may result in a device such as the IOP sensing device 400, though embodiments of method 900 may also result in IOP sensing devices 600 and 700 as depicted in FIGS. 6A-B and 7A-B, and other embodiments of such IOP sensing devices. At step 902, in order to fabricate an IOP sensing device 400, a substrate 402 is provided. The substrate 402 may be a printed circuit board, fabricated with layers of insulating plastic with electrical leads between and/or on the layers. The leads printed in between insulating layers may have electrical contacts disposed on the top most layer by which electrical connections may be made. The substrate 402 may be manufactured using semiconductor fabrication processes to create and insulate the electrical leads.

In some embodiments, the substrate 402 may include a chamber, and an inlet, and outlet, such as are depicted in FIG. 4C. These features may be manufactured using micromachining and/or semiconductor processing techniques. In some related embodiments, the membrane of the pressure sensor 409 may include piezoelectric elements by which pressure may be quantified for reference.

At step 904, a sensor, such as pressure sensor 408, may be coupled to the contacts so that power and signal lines may be provided between the sensor and a controller or processor. This may be accomplished by wire-bonding, through the inclusion of a ball grid array on the pressure sensor package, or any other suitable mechanism or structure. This may also be accomplished by fabricating the pressure sensor 408 into the substrate using microelectromechanical system (MEMS) fabrication techniques. At step 906, a pressure sensor cap 404 may be fixed or fabricated onto a top surface of the substrate 402 with an adhesive to form an interior chamber 406. As depicted, the pressure sensor cap 404 includes a wall structure 414A and a cap top 414B. In some embodiments the wall structure 414A is made from a semi-permeable material, such that gas may pass through the wall structure 414A while liquid may not. In other embodiments, the cap top 414B may provide the semi-permeable surface. Or in yet other embodiments, both the wall structure 414A and the cap top 414B may be made from a semi-permeable material or materials. At step 908, a flexible tube (not depicted), made of silicone or another suitable material, may be coupled to the inlet 410 of the chamber 406. The tube may be press fit around an attachment member 412, press fit into the inlet 410, adhesively fixed to the wall structure 414A, or otherwise attached to the pressure sensor cap 404. After the pressure sensor cap 404 is coupled to the substrate 402 and the tube, the IOP sensing device assembly may be encapsulated in a biocompatible material, such as PEEK or another biocompatible material such as, but not limited to, plastic, metal, glass, or silicon.

The systems and methods disclosed herein enable surgeons to more effectively remove all air from the pressure chambers by forcing the air through a semi-permeable surface that restricts passage of fluid. In particular, the semi-permeable chambers may facilitate the removal of gas bubbles that may adversely affect the accuracy of the pressure readings. This may result in more effective treatment and more accurate data, thereby improving the overall clinical result.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intraocular pressure (IOP) sensing device comprising:
a substrate comprising a substrate chamber, an inlet, and an outlet;
a differential pressure sensor formed from a flexible member located above the substrate, one side of the flexible member located adjacent the substrate chamber; and
a pressure sensor cap disposed on the substrate over the differential pressure sensor, the pressure sensor cap enclosing an interior chamber, the pressure sensor cap comprising a semi-permeable material.

2. The IOP sensing device of claim 1, wherein the pressure sensor cap is rectangular.

3. The IOP sensing device of claim 1, wherein pressure sensor cap is cylindrical, semispherical, elliptical, or ovoid.

4. The IOP sensing device of claim 1, wherein the pressure sensor cap is sealed to the substrate.

5. The IOP sensing device of claim 1, further comprising a tube coupled to the inlet.

6. The IOP sensing device of claim 5, wherein one end of the tube is configured to be placed in an anterior chamber of an eye

7. The IOP sensing device of claim 1, wherein the semi-permeable material is polytetrafluoroethylene.

8. The IOP sensing device of claim 1, wherein the interior chamber is pressurized thus exerting a force on the flexible member.

9. The IOP sensing device of claim 8, wherein the flexible member is displaced when a pressure in an anterior chamber of an eye is greater than a pressure in the interior chamber of the pressure cap.

10. The IOP sensing device of claim 8, wherein the flexible member is sealed against the substrate when a pressure in an anterior chamber of an eye is less than a pressure in the interior chamber of the pressure cap.

11. The IOP sensing device of claim 1 wherein the substrate chamber is fluidly coupled to the inlet.

12. The IOP sensing device of claim 1 further comprising:
a pressure sensor cap inlet located in the pressure sensor cap.

13. The IOP sensing device of claim 12 further comprising:
an attachment member surrounding a lumen, the lumen coextensive with the pressure sensor cap inlet.

14. The IOP sensing device of claim 13 further comprising:
a tube with proximal and distal ends, the proximal end coupled to the attachment member.

15. The IOP sensing device of claim 14 wherein the distal end of the tube is located outside of an anterior chamber of an eye.
